(19) United States
(12) Patent Application Publication
(10) Pub. No.: US 2004/0176315 A1
(43) Pub. Date: Sep. 9, 2004

(54) Methods of identifying optimal drug combinations and compositions thereof

(75) Inventor: Dale R. Pfost, Pennington, NJ (US)

Correspondence Address: MCDERMOTT, WILL & EMERY, 4370 LA JOLLA VILLAGE DRIVE, SUITE 700, SAN DIEGO, CA, 92122, US

(73) Assignee: ORCHID BIOSCIENCES, INC.

(21) Appl. No.: 10/797,164

(22) Filed: Mar. 9, 2004

Related U.S. Application Data
(62) Division of application No. 10/034,882, filed on Dec. 27, 2001.
Application No. 10/034,882 is a division of application No. 09/724,764, filed on Nov. 28, 2000, now abandoned.
(60) Provisional application No. 60/167,931, filed on Nov. 29, 1999.

(51) Int. Cl. A61K 48/00

(52) U.S. Cl. 514/44

(57) ABSTRACT

Provided are compositions of compounds effective for treating a pathology, the composition comprising at least two compounds that modulate the activity of one or more target molecules associated with one or more Single Nucleotide Polymorphisms (SNPs). Methods are also provided for increasing overall treatment efficacy for a population of patients having a pathology.

This application claims the benefit of U.S. Provisional Application No. 60/167,931, filed Nov. 29, 1999, which is incorporated herein by reference.

### BACKGROUND INFORMATION

The invention relates to compositions for effectively treating a population of patients having a pathology, and more specifically to compositions directed to effectively treat patients with a plurality of genetic profiles.

It has long been known that a pharmaceutical compound can be effective in treating certain patients afflicted with a pathology while being ineffective in treating other patients afflicted with the same pathology. Similarly, some patients with a pathology can experience adverse drug reactions as a result of the toxicity of the drug to those individuals, while others do not.

To improve the likelihood of effectively treating an individual, clinicians often simultaneously administer a variety of pharmaceutical compounds. Unfortunately, these pharmaceutical compounds often have very similar behavior and are not significantly more effective than any single compound, while potentially increasing the toxic effects experienced by the individual.

Accordingly, a need exists for novel compositions of pharmaceutical compounds which increase the likelihood of effectively treating an individual without resulting in toxic effects. The present invention satisfies this need and provides related advantages as well.

### SUMMARY OF THE INVENTION

The invention provides a composition of compounds effective for treating a pathology, the composition comprising at least two compounds that modulate the activity of one or more target molecules associated with one or more Single Nucleotide Polymorphisms (SNPs). The invention also provides methods of increasing overall treatment efficacy for a population of patients having a pathology, by analyzing compounds for efficacy correlated with the presence of a SNP associated with the target molecule and by selecting a combination of at least two of the compounds that exhibit the highest overall mean response of all dosing options in the population of patients.

### BRIEF DESCRIPTION OF THE FIGURES

FIG. 1 shows the response of patients having five different genotypes to different amounts of drug A, as described in Example I.

FIG. 2 shows the response of patients having five different genotypes to different amounts of drug B, as described in Example I.

FIG. 3 shows the response of patients having five different genotypes to different amounts of drug C, as described in Example I.

FIG. 4 shows the response of patients having five different genotypes to different amounts of drug D, as described in Example I.

FIG. 5 shows the response of patients having five different genotypes to different amounts of drug E, as described in Example I.

FIG. 6 shows the dose-response relationships of drug A in populations consisting of two genetic variants as described in Example II.

FIG. 7 shows the dose-response relationships of drug B in populations consisting of two genetic variants, as described in Example II.

### DETAILED DESCRIPTION OF THE INVENTION

In accordance with the present invention, there are provided compositions of compounds effective for treating a pathology, said composition comprising at least two compounds that modulate the activity of one or more target molecules associated with one or more Single Nucleotide Polymorphisms (SNPs), wherein each compound modulates the activity of at least one target molecule associated with one or more SNPs, and wherein said combination is effective for at least one patient having said pathology.

An invention composition increases the percentage of a patient population with a particular pathology that can be effectively treated relative to the use of individual compounds or relative to the use of known combinations of compounds on that population. The increased efficacy of the composition is achieved by combining compounds according to the efficacy of each compound for treating various subpopulations of patients with a pathology, in which each subpopulation has a unique set of genetic variations. Each subpopulation will typically have a unique characteristic response to therapeutic compounds. Knowledge of the efficacies of two or more compounds in treating patients with different genetic variations provides the opportunity to select the combination of compounds effective in treating a large percentage of the total population of patients while maintaining little or no toxicity.

The composition of the invention will comprise two or more compounds used to treat a pathology. Specifically, the phrase “two or more compounds” refers to a composition containing two, three, four, five, six, seven, eight, nine, ten or more compounds therein. As used herein, the term “compound” refers to a pharmaceutically active agent used to effect a physiological change in treating a pathology. Exemplary compounds can be chosen from drugs, pharmaceutically active natural products or dietary supplements, or any other type of compound (i.e., agent) useful in treating a pathology. Preferably, one or more of the compounds in the composition will be targeted toward treating a subset of the total population of patients with a pathology, in which the constituents of this subset have related or identical genetic profiles. A compound targeted toward such a subset of the total population refers to a compound directed to modulating the activity of one or more target molecules that play a role in the pathology, wherein the one or more target molecules are associated with one or more genetic variations characteristic of that subset of the population. Such a target molecule may play a role in the symptoms, etiology, side-effects, progression of treatment, and the like, of a pathology.

An invention composition of compounds preferably comprises specific amounts of two or more compounds, combined for the purpose of effectively treating an optimum percentage of patients with a pathology while maintaining little or no toxicity. Since a variety of different genetic variations can be associated with a single pathology, a composition can also include a combination of at least three compounds, a combination of at least four compounds, a combination of at least five compounds, a combination of at least six compounds, a combination of at least seven compounds, a combination of at least eight compounds, a combination of at least nine compounds, a combination of at least ten or more compounds, and the like. The amount of each compound used per dosage in an invention composition can vary from approximately 100 pg up to about 1 g (e.g., 100 pg, 1 ng, 10 ng, 100 ng or 1 mg, to 1 mg, 10 mg, 100 mg or 1 g, and the like).

As used herein, the phrase “effective against” refers to the effective exposure or contacting of a target molecule, or a cell or organism containing a target molecule, to a compound, such that such exposure or contacting is correlated with modulating the activity of the target molecule. This modulated activity will result in at least partial treatment of a pathology by, for example, alleviating symptoms of the pathology, treating the cause of the pathology, treating complications associated with a pathology, or otherwise effecting the treatment of a pathology, and the like. A compound of an invention composition can modulate the activity of a target molecule by, for example, increasing or decreasing enzymatic activity, increasing or decreasing gene expression, increasing or decreasing protein-protein interactions, increasing or decreasing signal transduction, increasing or decreasing transport or translocation across membranes, and the like. While a compound of an invention composition can modulate the activity of a target molecule by directly contacting the target molecule, it is also contemplated that a compound can also indirectly modulate the activity of a target molecule by contacting a different molecule that effects the activity of the target molecule.

The term “target molecule” refers to the molecule whose activity is modulated by a compound of the invention composition in the therapeutic action of the compound. A target molecule can vary from as large as an association of molecules, such as a ribosome or a lipid bilayer, to as small as a small molecule or an ion, such as a hormone, cytokine, cAMP, NO, Ca²⁺, K⁺, phosphate, and the like. An exemplary target molecule may be any molecule: (a) whose activity is modulated by the intended pharmacodynamic action of the compound; (b) involved in the absorption of a compound; (c) involved in the distribution of a compound within an organism., organ, tissue or cell; (d) involved in the biotransformation of a compound, such as the activation, degradation, and the like of a compound; or (e) involved in the excretion of a compound and/or metabolites of a compound. Typically, this target molecule will be a biological macromolecule, such as RNA, DNA or a protein. In one embodiment, a preferred target molecule is a protein. Preferably, a target molecule is a protein whose activity is modulated by a compound in the intended pharmacodynamic action of the compound.

A compound that is effective against a target molecule associated with one or more particular genetic variations refers to the ability of a compound to modulate the activity of a target molecule that plays a role in the symptoms, etiology, complications or treatment of a pathology. Preferably, a compound modulates the activity of a target-protein associated with one or more genetic variations that plays a role in the symptoms, etiology, complications or treatment of a pathology. For example, a protease normally having a glutamate at a position near the active site can have increased proteolytic activity as a result of a single nucleotide polymorphism arising in which the glutamate is changed to alanine (by, for example, an A→C nucleotide polymorphism), resulting in this single nucleotide polymorphism playing a role in a pathology caused by increased proteolytic activity. A compound, such as a protease inhibitor, can be effective against a protease associated with this single nucleotide polymorphism by inhibiting the proteolytic activity of the protease.

As used herein, a “genetic variation” refers to any deletion, insertion or base substitution of the genomic DNA of one or more individuals in a first portion of a total population which thereby results in a difference at the site of the deletion, insertion or base substitution relative to one or more individuals in a second portion of the total population. Thus, the term “genetic variation” encompasses “wild type” or the most frequently occurring variation, and also includes “mutant,” or the less frequently occurring variation. A preferred type of genetic variation is a single nucleotide polymorphism, or SNP. As used herein, a SNP refers to a genetic variation at a specific site in the genome of an organism, where the nucleotide identity at that site varies between genomic allelic members of a population of organisms. In one embodiment of the present invention, a genetic variation or a SNP that is correlated with a particular pathology is known to effect: an individual's predisposition to acquire that pathology; the severity of the pathology in an individual; an individual's response to therapeutic treatment of that pathology; and the like. Typically, the efficacy and/or toxicity of a compound is correlated with one or more genetic variations or SNPs or the expression of one or more SNPs. Accordingly, it is contemplated herein that a compound interacts with a target molecule at a position on the target molecule corresponding to one or more SNPs, or the expression of one or more SNPs. Preferably, the mechanism of action of a compound on a target molecule having one or more genetic variations or SNPs is known.

As used herein, a target molecule that is “associated with” or “correlates with” a particular genetic variation, preferably a particular SNP, is a molecule that can be functionally distinguished in its structure, activity, concentration, compartmentalization, degradation, secretion, and the like, as a result of such genetic variation.

In a particular pathology, it is contemplated herein that one or more genetic variations, preferably one or more SNPs, can be correlated with the symptoms, etiology, side-effects or progression of treatment of that pathology. Additionally, one or more genetic variations can be correlated with the efficacy, or toxicity, or both, of a compound used for treating the pathology. As used herein, the “correlation of a genetic variation with a pathology” refers to the increased occurrence of the pathology in a first portion of a total population having a particular genetic variation relative to a second portion of a total population having a second genetic variation. As used herein the “correlation of a genetic variation with effective treatment” of a pathology refers to an increased efficacy of treatment of the pathology in a first portion of a total population having a particular genetic variation relative to a second portion of a total population having a second genetic variation. Patients with a particular pathology can have a wide variety of one or more genetic variations, all of which are correlated with that pathology. A particular combination of genetic variations correlated with a pathology or the treatment of the pathology is referred to as a genetic profile or a genotype. Thus, a patient or group of patients with a pathology that have the same combination of genetic variations correlated to that pathology can be characterized as having a particular genetic profile.

In addition, a patient or group of patients with a particular pathology that have similar combinations of genetic variations can be characterized as having a similar genetic profile. As used herein, a group of patients with a “similar genetic profile” refers to a group of patients where each member of the group has at least 50% of the same genetic variations correlated to a particular pathology as every other member of the group. Preferably, a group of patients with a similar genetic profile has at least 60% of the same genetic variations as every other member of the group. More preferably, a group of patients with a similar genetic profile has at least 75% of the same genetic variations as every other member of the group. Most preferably, a group of patients with a similar genetic profile has at least 90% of the same genetic variations as every other member of the group.

A genetic variation, such as a SNP, can be identified by finding a difference in the nucleotide sequence of an individual compared to the most common nucleotide sequence of the overall population. Methods used to identify genetic variations, such as SNPs, are well known in the art and include hybridization stability methods such as SSCP, where the hybrids are identified, for example, by electrophoretic analysis, denaturing HPLC (U.S. Pat. No. 5,795,976), or addressable DNA array hybridization (U.S. Pat. No. 5,547,839). The perturbation resulting from the hybrid instability can be exploited to detect SNPs by its impact on enzymatic reactions such as restriction endonucleases (RFLP) (U.S. Pat. No. 4,623,619), allele-specific oligonucleotide ligation (U.S. Pat. No. 4,988,617), allele-specific cleavage (U.S. Pat. No. 5,422,253), allele-specific PCR (U.S. Pat. No. 4,683,195), and allele-specific LCR (U.S. Pat. No. 5,494,810). Other methods for detecting SNP genetic variations use polymerase-dependent primer extension techniques such as GBA which uses single nucleotide extension (U.S. Pat. No. 5,888,819) or limited extension from a specific primer for analysis by, for example, mass spectrometry (U.S. Pat. No. 5,547,835). Correlation of data to identify a site of a genetic variation such as a SNP can be carried out by sequence comparison of the results of the above experiments for multiple individuals (Nickerson et al., Nucleic Acids Res 25:2745-2751 (1997), U.S. Pat. No. 5,762,876).

In accordance with the present invention, certain genetic variations are correlated with a pathology or treatment of a pathology. For example, the SNP encoding the change from normal hemoglobin to sickle hemoglobin in sickle cell anemia. Methods for using a variety of patient determinants such as genetic variations to establish if one or more determinants are correlated with a pathology, or if one or more determinants are correlated with treatment of a pathology are known in the art and are exemplified in U.S. Pat. No. 5,860,917 and in publications such as WO 97/13875, WO 97/21833, WO 99/11822, WO 99/24571, each of which is incorporated herein by reference.

In addition to genetic variations in a patient population, it is also contemplated herein that other determinants such as diet, exercise, smoking, prior medical history, and the like, can also be correlated to a pathology or treatment of a pathology by a compound of the invention. These further determinants are particularly useful when used in concert with one or more genetic variants in finding the correlation of such determinants with a pathology or treatment of a pathology.

A compound that is “stably effective” for a particular percentage of a total patient population is a compound for which the effectiveness does not change over a time scale of 1, 2, 5, 10, 25., 50 or 100 years. For example, a therapeutic compound used to treat a viral pathology will often lose its effectiveness after a few years as a result of the evolutionary changes undergone in the viral genome, and thus will typically not be stably effective. However, a pathology such as a human genetic disorder, does not change quickly over time. Therefore, a compound effective for treating such a genetic disorder will be effective over a long period of time, and thus is considered stably effective in treating that pathology.

As used herein, a total patient population is a group of patients which each have a particular pathology. For example, a total patient population can be all patients having glaucoma. In addition, a total patient population can be a subset of a larger population of patients. For example, a subset population of glaucoma patients can be all patients suffering from pigmentary glaucoma. In this case, the total patient population is a population in which each patient has pigmentary glaucoma. Thus a total patient population can be any group of patients having a common pathology, or further can be any group of patients having a common pathology and also having similar or identical symptoms, diagnostic markers, medical history, genetic profile, or the like.

The composition of the invention will be effective for at least one patient having a particular pathology. Preferably, the composition of the invention is effective for at least 1% of a subset of the total patient population, where the subset population is defined as a group of patients smaller than the total population and the members of the subset population have similar or identical symptoms, diagnostic markers, medical history, genetic profile, or the like.

In one embodiment of the invention, the composition is effective for a greater percentage of the total patient population than any single compound. Typically, such a composition is effective for at least 10%, 15%, 20%, or 25% of the total patient population. Preferably, the composition is effective for at least 50% of the total patient population. More preferably, the composition is effective for at least 75% of the total patient population. Most preferably, the composition is effective for at least 90% of the total patient population.

The percentage of the total patient population for which an invention composition is effective can be calculated. Typically, the first step in calculating the percent efficacy of a composition is determining the percentage of each subpopulation (i.e., group of patients having the same genetic profile) for which a particular amount of each compound of the invention composition is effective per dose. Next, the percent efficacy of a composition of two or more compounds is determined for each subpopulation. Finally, the percent efficacies for all subpopulations are combined to result in an overall percent efficacy for the total patient population.

Calculation of the percentage of a subpopulation for which a particular amount of a compound is effective per dose is carried out by correlating the efficacy of one or more particular amounts of that compound in treating a particular percentage of one or more subpopulations of a patient population. In one embodiment, this calculation includes consideration of the amount of compound necessary to produce a therapeutic effect, as described by the ED₅₀. As used herein, ED₅₀ refers to the dosage Effective Dosage to treat 50% of patients having a particular genetic profile. Thus a therapeutic compound effective against a pathology only for a subpopulation of patients with a particular genetic profile which make up 20% of the total patient population will be effective for 10% of the total patient population when administered at a dosage level of ED₅₀=1.

In one embodiment of the invention, the level of efficacy of a compound for a subpopulation of the total patient population is determined by the one or more target molecules (e.g., one or more target-proteins) against which the compound is effective, and by the genetic variations that are correlated with the efficacy of the compound against the one or more target molecules. Typically, a compound will be more effective for at least one subpopulation than for at least one other subpopulation. For example, a particular compound may be highly effective against a target molecule in a subpopulation of patients having a particular genetic profile, while that compound may be ineffective against that target molecule in any other subpopulation of patients having a different genetic profile.

A therapeutic compound that causes a toxic reaction in patients with a particular pathology is characterized as toxic for a particular percentage of the total patient population. The percentage of the total patient population for which a therapeutic compound is toxic per dose is typically determined by the amount of the compound necessary to produce a toxic effect for each subpopulation, having a particular genetic profile, of the total patient population.

In another embodiment of the invention, a composition of compounds is provided which is targeted against at least one target molecule associated with a pathology, the composition comprising at least two compounds that each are effective against at least one genetic variation associated with the target molecule(s), in which the combination is stably effective for at least one patient having the pathology. In this embodiment, it is possible for each compound to be effective against the same genetic variation within the same target molecule, while the efficacy of each compound against that genetic variation can be the same or can be different. It is also within the scope of this embodiment that each compound is effective against mutually exclusive genetic variations (e.g., SNPs) within the same target molecule. For example, a first compound can be effective against a SNP in the active site of a protein by acting as an enzymatic inhibitor, while a second compound, such as a monoclonal antibody, can be effective against a surface-residue SNP on the same protein. In both of the above possibilities, two or more compounds effective against genetic variation(s) of a single target molecule allow the two or more compounds to have complementary efficacies against the same protein, thus resulting in an increased efficacy against the target molecule, relative to the efficacy of any single compound.

It is also within the scope of this embodiment that the composition of two or more compounds is effective against genetic variations associated with at least two target molecules. For example, in an apoptotic disease involving an altered level of activation of a caspase by a cytokine, a first compound can be effective against a SNP of the cytokine receptor, while a second compound can be effective against a SNP of the caspase. A combination of these two compounds will therefore be effective for a combination of the populations for which each single compound is effective. Further, the invention composition of two or more compounds can be effective against at least one, at least two, at least three or more of these target molecules, each of which are associated with at least two genetic variations.

In yet another embodiment of the invention, an invention composition of compounds is provided in which the efficacies of the compounds are correlated with modulating the activity of a single target-protein associated with a pathology. This particular invention composition comprises at least two compounds that are effective in treating patients having one or more SNPs within the target-protein, wherein the combination is effective for at least one patient having said pathology. In this embodiment, each compound can be effective in treating patients having the same SNP associated with a single target molecule, or each compound can be effective in treating patients having mutually exclusive SNPs associated with a single target protein.

Thus, for a pathology in which one protein associated with the pathology can be one of a plurality of different genetic variants, a composition of the invention can be directed toward treating the various groups of patients having these different variants. For example, three different forms of the β-adrenergic receptor can exist in patients who have suffered congestive heart failure. A composition of the invention can comprise three compounds that block β-adrenergic receptor activity. (β-blockers), in which each compound is effective for a different form of the adrenergic receptor.

In another embodiment of the invention, a method is provided for increasing treatment efficacy for a given population of patients having a pathology, comprising:

(a) analyzing compounds known or suspected of acting on at least one target-protein corresponding to the pathology for efficacy correlated with the presence of a genetic variation on the target protein(s); and

(b) selecting, for treatment of the patients, a combination of at least two of the compounds that exhibits the highest overall mean response in treating the population of patients.

As used herein, the phrase “increasing treatment efficacy” or “increasing treatment coverage” and grammatical variants thereof, refers to increasing the percentage of a given population of patients having a pathology for which a combination of compounds is effective. A method of increasing treatment efficacy will be influenced by a variety of factors. The principle factors determining the treatment efficacy by a particular composition of two or more compounds are: percentage of coverage by each individual compound, interaction (e.g., drug overlap) between different compounds in a composition, and toxicity restrictions.

As discussed herein above, compounds known or suspected of modulating the activity of at least one target molecule can be analyzed for the correlation of the efficacy of the compound with one or more genetic variations in patients with a pathology. This efficacy can be represented as the percentage of therapeutic coverage (i.e., therapeutic effectiveness) for particular compound, which is determined by the percentage of therapeutic coverage of each of the subpopulations of a patient population in conjunction with the amount of the compound present per dose of the combination.

Once the treatment efficacies of two or more individual compounds have been correlated with different genetic variations (e.g., SNPs), a combination of compounds can then be selected according to the desired treatment efficacy of a patient population that is achieved by such a combination of the therapeutic compounds.

Overall treatment efficacy of a patient population by an invention composition will be affected by the interaction of the two or more compounds in the composition. For example, if two compounds which are directed to the same target molecule and are effective against the same SNPs of the target molecule in the same way, a considerable amount of drug overlap can result. Complete drug overlap, as opposed to partial drug overlap, will limit the extent of increased treatment efficacy resultant from combining two compounds, as the efficacy of the combination of compounds can represent no more than the coverage provided by one of the compounds.

Alternatively, a combination of compounds in which each compound is independently effective against a different genetic variation can result in the efficacy of the combination being equal to the sum of the efficacies of the individual compounds. For example, in the simple case in which all patients with a particular pathology have one of two SNPs, a particular amount of a first compound is effective for 90% of a first subpopulation forming 50% of the total patient population, is combined with a second compound effective for 60% of a second subpopulation forming the other 50% of the total patient population, the treatment efficacy for of the total patient population will be 75% (50%×90%+50%×60%). In such an example, whereas neither compound is effective for even half of the total patient population individually, a combination of the two compounds is effective for a majority of the total patient population.

The efficacy of a combination of compounds can also be greater than the sum of individual efficacies. For example, patients with a particular pathology can have either one or two SNPs, and the total patient population is distributed in such a way that 33% of the total patient population has a first SNP, 33% of the total patient population has a second SNP, and 33% of the total patient population has both the first and second SNPs. If a first compound is effective for 90% of the total patient population having the only the first SNP, and a second compound is effective for 60% of the total patient population having the only the second SNP, the summed efficacy would be 50% (33%×90%+33%×60%). However, the combination, being also effective against the population having both SNPs, would have an efficacy of 68% (33%×90%+33%×60%+33%×60%×90%).

In addition to relationships described hereinabove for interaction between compounds effective against genetic variations when used in combination, those of skill in the art will recognize that other relationships exist which can result in a variety of effects when combined, such as: two or more compounds being effective against a particular genetic profile or subpopulation of patients against which no compound is individually effective; two or more compounds having partial overlap between subpopulations; a first compound enhancing the efficacy of a second compound against a particular subpopulation against which the first compound alone has no efficacy and the second compound alone has relatively low efficacy; and the like.

Irrespective of the particular mechanism involved, two or more compounds will have a relationship that is either: 1) completely overlapping, resulting in no increased or decreased efficacy relative to one or more compounds in the composition; 2) independent, resulting in additive efficacies in the composition; 3) partially overlapping, resulting in some additive effect, but less than that observed in the independent relationship; 4) canceling, resulting in efficacy below that of at least one compound in the composition; or 5) enhancing or synergistic, resulting in an efficacy of the composition being greater than the sum of the efficacies of each compound in the composition.

Toxicity will also influence treatment coverage by a particular composition of two or more compounds. While each compound will have a corresponding level of toxicity when individually administered to a patient, combinations of compounds can have toxicity interactions in a manner similar to combinations of compounds having efficacy interactions, resulting in different toxicities for different combinations of compounds. These toxicity relationships can be overlapping, independent, enhancing or canceling. However, for compound toxicity, an overlapping relationship will result in additive toxicities of the individual compounds, while independent relationship will result in no additive or decreased toxicity relative to the individual compounds. Typically, the relationship between toxicities of two or more compounds will be overlapping or additive, as determined by summing the percentages of threshold level of toxicity for each compound for a single dose. For example, if a first compound has a threshold toxicity of 100 mg, and a single dose has. 10 mg of this compound, the dosage of this compound is 10% threshold toxicity. If the first compound is in a composition with 10 mg of a second compound, where the second compound has a threshold toxicity of 20 mg (the second compound therefore being at 50% toxicity), the total toxicity for the composition will be 60% threshold toxicity (10%+50%).

Non-interacting toxicities will not be additive, but considered separately. Thus a composition of two compounds, each administered at 90% individual threshold toxicity, is not above threshold toxicity since neither compound is toxic on its own, nor does either compound add to the toxicity of the other. Toxicities that enhance one another will result in an overall toxicity higher than expected by summing the individual toxicities. Toxicities that cancel one another will result in an overall toxicity lower than expected from the toxicity of one or more individual compounds.

In a specific embodiment of the invention, the toxicity of a compound or of a composition of two or more compounds is different for different subpopulations within a population of patients. In this instance, a step of formulating a composition that has little or no toxicity for the entire population includes determining the toxicity of a composition for each different subpopulation. Particular subpopulations, for example, can have a genetic variation (e.g., a SNP) in an enzyme used for breaking down one or more compounds in the composition, this genetic variation resulting in modulated toxicity relative to the subpopulations that do not have this genetic variation.

As used herein, “toxicity” and grammatical variants thereof refers to a harmful effect caused by a compound in a role other than its intended pharmaceutical role, resulting in discomfort or endangerment of the patient. Typically a toxic effect of a compound will cause necrosis or other permanent or irreversible damage to a patient. The dosage level considered to be minimally toxic or to have no toxicity is a dosage level that is not toxic to even a single member a total population of patients. A dosage level of a compound having threshold toxicity is considered a dosage level at which the compound is toxic to at most 10% of a population, or alternatively is considered a dosage level at which a 20% increase in dosage level results in a 2-fold increase in the percent of a population for which the compound is toxic. Preferably, a compound will be administered at a dosage level that is toxic to less than 5% of a total patient population and, more preferably, less than 1% of a total patient population.

Methods are known for analyzing compounds known or suspected of acting on at least one target molecule corresponding to the pathology for efficacy correlated with the presence of a genetic variation associated with a target molecule. Analysis of compounds for their ability to act on a target molecule can be carried out using a wide variety of methods, including in vivo, in vitro and in situ binding and/or inhibition assays, in vivo survival assays, clinical trials, and any other method useful to correlate the ability of a compound to modulate the activity of a target molecule (see, for example, Current Protocols in Molecular Biology (John Wiley and Sons, NY), which is incorporated herein by reference). A compound found to modulate the activity of a target molecule can then be further tested to determine the efficacy of this compound in treating a pathology in each subpopulation of a patient population. Such testing methods can be carried out using suitable animal models, or clinical trials. Similarly, such a compound can be tested to determine its toxicity in the various subpopulations of a patient population.

The process of selecting a combination of two or more compounds from all dosing options to optimize therapeutic coverage of the total patient population preferably includes: selection of combinations of compounds that are effective for a large percentage of a patient population, such as a combination having the highest overall mean response; and selection of combinations that have little or no toxicity for all of the total patient population. Suitable combinations of compounds will be combinations which are effective for one patient, at least 1% of the total patient population, at least 5%, 10%, 15%, 20%, at least 25% of the total patient population, preferably effective for at least 50% of the total patient population, more preferably effective for at least 75% of the total patient population, most preferably for at least 90%, 95% or 98% of the total patient population. Typically, the combination selected will be effective against the maximum percentage of the total patient population while maintaining little or no toxicity.

The term “highest overall mean response” refers to the combination of compounds which have the highest percent efficacy for a total patient population. Typically, a combination with the highest overall mean response is above threshold toxicity for no more than 25% of a total patient population, preferably less than 15% of a total patient population, more preferably less than 10% of a total patient population, even more preferably less than 5% of a total patient population, most preferably no more than 1% of a total patient population. It is also contemplated herein that an invention composition will not have any toxicity for any portion of the patient population (e.g. 0% toxicity).

In accordance with another embodiment of the present invention, there are provided methods of determining a composition that has the highest mean response for a group of patients representing less than the total population. Such a sub-population may be a single patient, or may be a group of patients with identical or similar genetic profile, or may be several groups of patients with identical or similar genetic profiles.

In another embodiment of the invention, a method is provided for maximizing overall population treatment efficacy of treatment for a particular pathology, comprising:

(a) determining the efficacies of a plurality of compounds known or suspected of treating said pathology;

(b) determining the toxicity of said plurality of compounds; and

(c) selecting from said plurality of compounds a combination of compounds that is minimally toxic and is stably effective for at least one patient having said pathology.

Another embodiment of the invention provides a genotypically-facilitated method of treating a patient having a pathology comprising:

(a) analyzing a therapeutic target molecule in a population of patients having said pathology to detect genetic variations, preferably SNPs, associated therewith;

(b) selecting a plurality of compounds having therapeutic efficacies correlated with the presence of at least one genetic variation associated with a target-protein; and

(c) administering said plurality of compounds to said patient;

wherein said combination is effective for at least one patient having said pathology.

The step of administering a plurality of compounds to a patient is carried out by any of a variety of methods known in the art. For example, it is within the scope of this invention that the plurality of compounds are administered to a patient as a pill, inhalant, topical application, suppository, by intravenous infusion, or any other mode well-known in the art for introducing a therapeutic compound or composition to a patient.

The step of administering a plurality of compounds can be carried out in such a way that two or more compounds are administered simultaneously, either in a single pharmaceutical composition of two or more compounds or as two or more separate pharmaceutical compositions of two or more compounds. It is further within the scope of the invention that the plurality of compounds are administered to a patient proximately. By “proximate administration” of a plurality of compounds and grammatic variants thereof is meant administration of at least two of a plurality of compounds non-simultaneously, but in close temporal proximity. For example, a first compound can be administered to a patient to reduce the swelling in a joint, followed by the administration of a second compound to reduce cytokine activity in the joint, and the like.

Typically, two or more compounds so administered proximately will be administered within 72 hours of each other. Preferably, such compounds will be administered within 24 hours of each other. More preferably, such compounds will be administered within 6 hours of each other. Most preferably, such compounds will be administered within 1% hour of each other.

Provided in a further embodiment of the invention is a method of formulating a pharmaceutical composition for treating a particular pathology in a population of patients having said pathology, comprising:

(a) measuring a correlation of genetic variation of a target molecule in said population with patient response to at least one compound known or suspected to treat said pathology; and

(b) selecting at least two compounds that provide the greatest percentage of efficacy in said patient population, wherein said percentage is at least 1% of patients having said pathology.

Percentages of efficacy also contemplated herein include is at least 5%, at least 10%, at least 15%, at least 20%, at least 25%, at least 50%, at least 75%, at least 90%, at least 95% or at least 98%.

Another embodiment of the invention provides a method of formulating a therapeutic composition to treat a pathology in a population of patients having the pathology, comprising:

(a) analyzing a target molecule in a patient population to detect one or more genetic variations, preferably SNPs, associated therewith; and

(b) selecting a plurality of compounds having therapeutic efficacies correlated with the presence of at least one genetic variation associated with the target molecule, wherein said plurality is effective for at least one patient having said pathology.

A formulated composition of the invention will be a combination of two or more selected compounds, specifying the amount of each selected compound, to be used per dose. This composition will typically be formulated such that it is either: effective for a maximum percentage of a total patient population; or exhibits the lowest variation in efficacy across different subpopulations of patients having the same genetic profile (e.g. an SNP profile); or both.

A composition that exhibits the lowest variation in efficacy across different subpopulations of patients having the same genetic profile (e.g., a SNP profile, and the like) refers to a composition that most closely approaches having equal efficacy for each patient subpopulation. For example, a first composition that is effective for 75% of a first subpopulation and effective for 65% of a second subpopulation exhibits a lower variation than a second composition that is effective for 95% of a first subpopulation and 45% of a second subpopulation. In the above example, if the two subpopulations represent 50% of the total patient population, the first and second compositions will be effective for the same overall percentage of the total patient population, but the first composition can be a preferred composition when it is desired to formulate the invention composition to have the lowest variation across different subpopulations.

In a further embodiment of the invention, a method is provided for minimizing the toxicity of a combination of compounds effective in treating a pathology. It is within the scope of the invention that a plurality of combinations of compounds can have overall mean efficacies within 25% or within 10% of each other, while the toxicities of such combinations can vary by more than 10% or more than 20%. When such a plurality of combinations exist, a combination which has the lowest toxicity can be preferred in favor of another combination which has a higher overall mean efficacy.

A composition of the invention comprises two or more compounds effective for at least one patient having a pathology. In addition to compounds effective for at least one patient having a pathology, a composition can also comprise a pharmaceutically acceptable carrier.

As used herein, the terms “pharmaceutically acceptable”, “physiologically compatible” and grammatical variations thereof, as they refer to compositions, carriers, diluents and reagents, are used interchangeably and represent that the materials are capable of administration to a mammal without the production of undesirable physiological effects such as nausea, dizziness, gastric upset, and the like. Examples of pharmaceutically acceptable carriers are phosphate buffered saline solution, water and emulsions such as an oil/water or water/oil emulsion, and various types of wetting agents

The preparation of a composition that contains active ingredients dissolved or dispersed therein is well known in the art. Typically such compositions are prepared as injectables either as liquid solutions or suspensions; however, solid forms suitable for solution, or suspension, in liquid prior to use can also be prepared. The preparation can also be emulsified.

The active ingredient can be mixed with excipients which are pharmaceutically acceptable and compatible with the active ingredient in amounts suitable for use in the therapeutic methods described herein. Suitable excipients are, for example, water, saline, dextrose, glycerol, ethanol, or the like, as well as combinations of any two or more thereof. In addition, if desired, the composition can contain minor amounts of auxiliary substances such as wetting or emulsifying agents, pH buffering agents, and the like, which enhance the effectiveness of the active ingredient.

The therapeutic composition of the present invention can include pharmaceutically acceptable salts of the components therein. Pharmaceutically acceptable nontoxic salts include the acid addition salts (formed with the free amino groups of the polypeptide) that are formed with inorganic acids such as, for example, hydrochloric acid, hydrobromic acid., perchloric acid, nitric acid, thiocyanic acid, sulfuric acid, phosphoric acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, and the like.

Salts formed with the free carboxyl groups can also be derived from inorganic bases such as, for example, sodium hydroxide, ammonium hydroxide, potassium hydroxide, and the like; and organic bases such as mono-, di-, and tri-alkyl and -aryl amines (e.g., triethylamine, diisopropyl amine, methyl amine, dimethyl amine, and the like) and optionally substituted ethanolamines (e.g., ethanolamine, diethanolamine, and the like).

Physiologically tolerable carriers are well known in the art. Exemplary liquid carriers are sterile aqueous solutions that contain no materials in addition to the active ingredients and water, or contain a buffer such as sodium phosphate at physiological pH, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary additional liquid phases include glycerin, vegetable oils such as cottonseed oil, and water-oil emulsions.

In another embodiment of the invention, a method is provided for preparing a combination of compounds for treating one or more patients having a pathology, wherein said combination of compounds has increased efficacy and/or reduced toxicity, relative to any individual compound, in a greater portion of a population of patients having said pathology, comprising:

(a) correlating the efficacy and/or toxicity of a first compound with the presence of one or more genetic variations;

(b) correlating the efficacy and/or toxicity of a second compound with the presence of one or more genetic variations; and

(c) calculating the efficacy and/or toxicity of a combination of said first compound and said second compound on said population of patients;

wherein said combination is effective for at least one patient having said pathology. It is also contemplated herein that the combination of compounds can be effective for at least 5%, at least 10%, at least 15%, at least 20%, at least 25%, at least 50%, at least 75%, at least 90%, at least 95% or at least 98% of the total patient population.

A method of correlating efficacy or toxicity of a compound with one or more genetic variations as disclosed herein and as known in the art can be individually carried out for at least two compounds, and will typically be carried out for more than two compounds. For example, such a method can be carried out for 3, 4, 5, 6, 7, 8, 9, or for 10 or more compounds. The efficacies or toxicities of combinations of such a plurality of compounds can then be calculated.

Also provided herein is an algorithm for determining the efficacy and/or toxicity of a combination of two or more compounds for a population of patients having a pathology, comprising the steps of:

(a) correlating the efficacy and/or toxicity of each of said two or more compounds with the presence of one or more genetic variations, preferably SNPs; and

(b) combining the correlations of the efficacy and/or toxicity of each of said two or more compounds with the presence of one or more genetic variations;

whereby the efficacy and/or toxicity of said combination of compounds for said population of patients is calculated.

The step of combining the correlations of the efficacy or toxicity of each of two or more compounds is carried out in accordance with the interaction of each compound with the other compounds. As disclosed herein, such interaction can be overlapping, independent, partially overlapping, canceling or enhancing. Correlations so combined will result in a predicted efficacy or toxicity of one or more combinations of compounds in treating a population of patients with a pathology, and will additionally result in predicted efficacies or toxicities of one or more combinations of compounds in one or more subpopulations of patients, where a subpopulation of patients is a group of defined as patients having the same genetic profile with respect to a pathology.

In a further embodiment of the invention, a method is provided for treating one or more patients having a pathology with a combination of therapeutic compounds having increased efficacy and/or reduced toxicity, relative to any individual compound, in a greater portion of a population of patients having said pathology, comprising the steps of:

(a) correlating the efficacy and/or toxicity for each of two or more compounds with one or more genetic variations, preferably SNPs;

(b) calculating the efficacy and/or toxicity of administering said two or more compounds on said population of patients; and

(c) administering said two or more compounds to said patients;

wherein said two or more compounds are effective for at least one patient having said pathology. It is also contemplated herein that the two or more compounds can be effective for at least 5%, at least 10%, at least 15%, at least 20%, at least 25%, at least 50%, at least 75%, at least 90%, at least 95% or at least. 98% of the total patient population.

In a further embodiment, one or more compounds of a composition can be differently effective against one genetic variant than another. For example, a SNP-associated variant of a protease can arise that results in a glutamate changing to an alanine. Use of a protease inhibitor compound can result in the inhibition of the alanine SNP variant without inhibiting the glutamate SNP variant. Thus, it is within the scope of this invention that a compound can have a unique efficacy against each SNP variant of a target molecule.

However, it is also within the scope of the invention that a compound can have identical efficacies for one or more SNP variants of a target molecule. For example, a monoclonal antibody targeted against a protease can bind a region distant from the location of the SNP residue, thus the efficacy of the antibody will not be influenced by the presence of different SNP variants of the target protease.

Further, a compound can be equally effective against particular SNP variants, but differently effective against others. For example, a protease inhibitor, effective against a protease having three SNP variants, tryptophan, aspartate and asparagine, at a particular position can have the same efficacy against the aspartate and asparagine variants, but a different efficacy against the tryptophan variant.

The invention will now be described in greater detail by reference to the following non-limiting example. All U.S. patents and all publications mentioned herein are incorporated in their entirety by reference thereto.

### EXAMPLES

### Example I

Five Drugs with Additive Efficacies for Five Equally Populated Genotypes

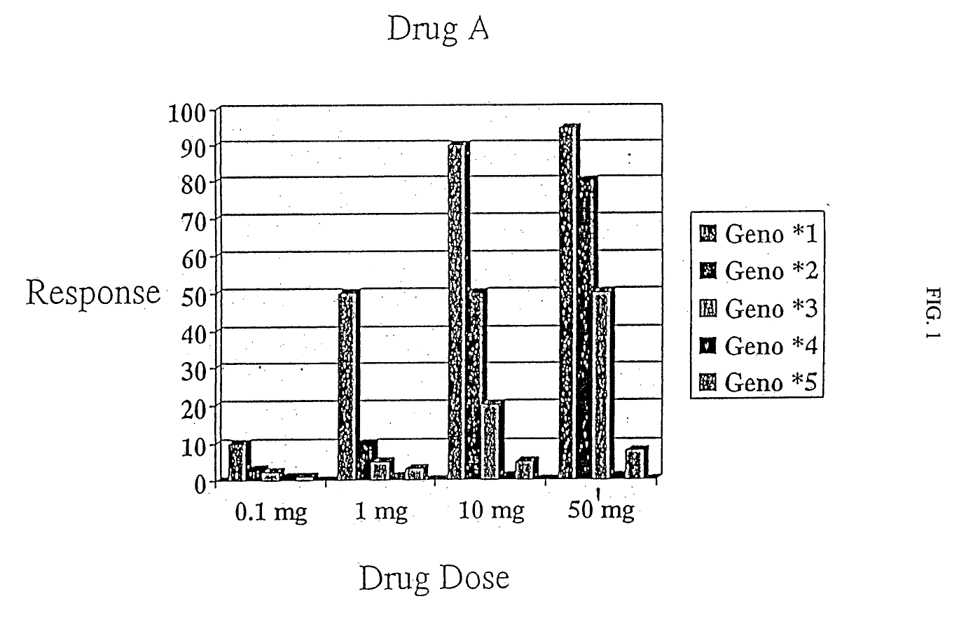

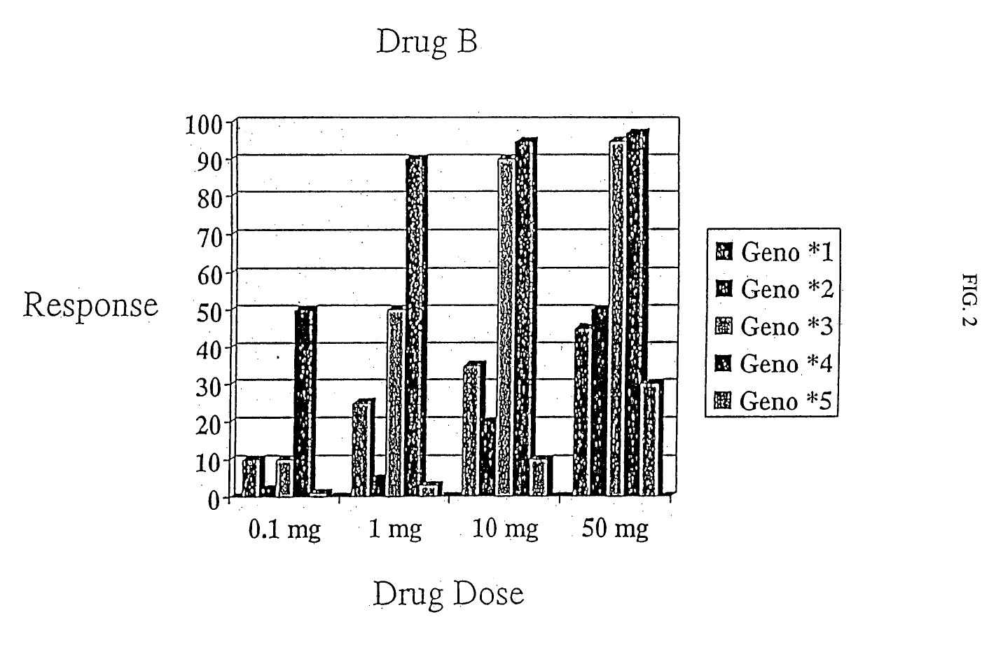

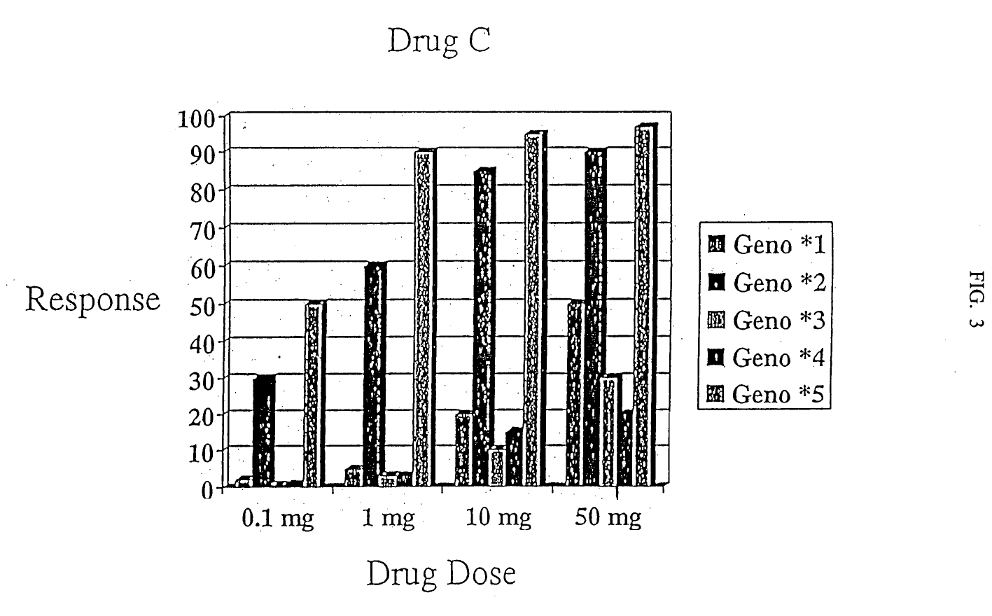

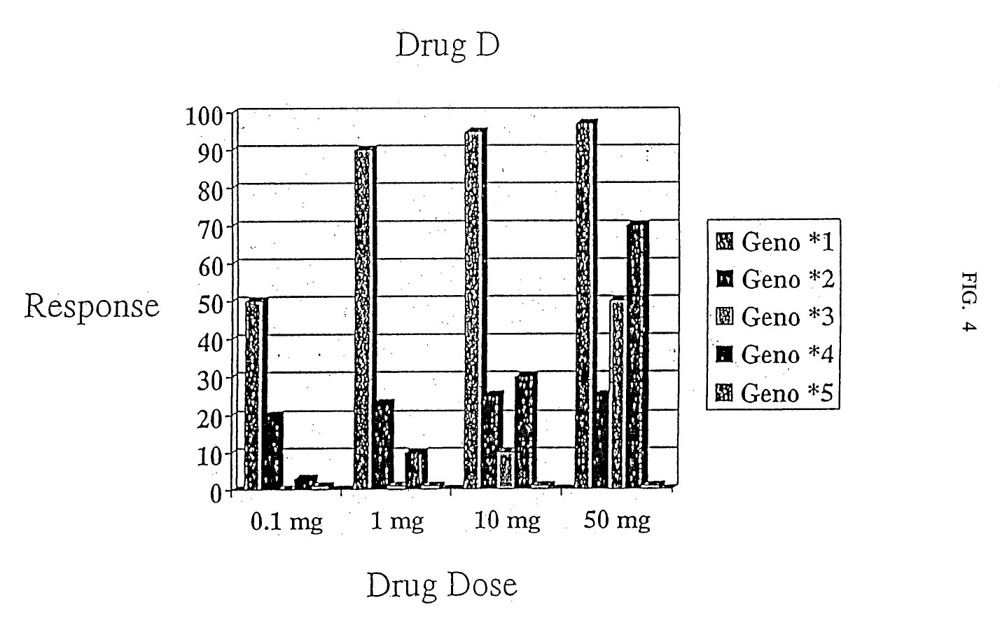

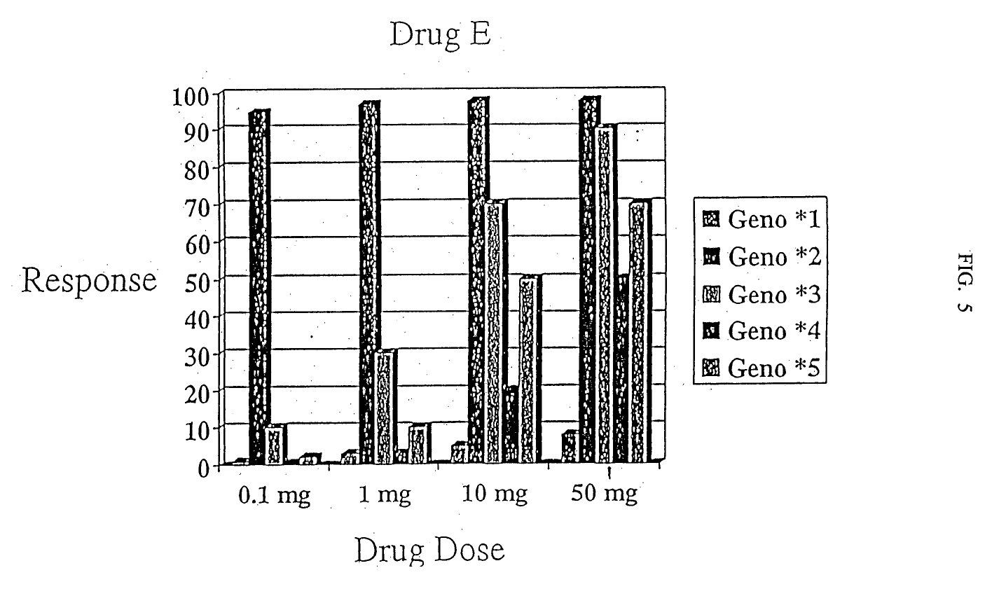

A variety of drugs known to treat a pathology can be selected for analysis in order to determine a suitable combination of drugs for treating the pathology. For example, drugs A, B, C, D and E which are all used to treat a particular pathology can be subjected to the following analysis. Each of the five drugs has a characteristic dose-response curve that is different for each of five different equally populated genotypes: *1, *2, *3, *4 and *5. Dose-response curves are shown for drugs A, B, C, D and E in FIGS. 1, 2, 3, 4 and 5, respectively. Assuming each drug acts on a population or subpopulation in a manner that is independent from each of the other drugs, the efficacies of multiple drugs are additive. For example, if drugs A and B act independently of each other with respect to the subpopulation of patients in genotype *1, then the 10% of patients in genotype *1 effectively treated by 0.1 mg of drug A, can be added to the 10% of patients in genotype *1 that are effectively treated by 0.1 mg of drug B, resulting in 20% of patients in genotype *1 being effectively treated by a combination of 0.1 mg drug A and 0.1 mg drug B (see Table I).

In this manner, optimized combinations of drugs can be designed which treat a large percentage of the overall population of patients with a pathology, by adding together the percentages of each genotype treated by particular combinations of drugs at a particular dosages.

Once the percentage of a particular genotype exceeds 100%, no additional drugs or larger dosages are beneficial to that genotype. Thus, use of a combination of two drugs that are effective in treating the same genotype or genotypes, will have only a limited increased efficacy for the entire population. For example, if drugs D and E are combined at 0.1 mg each, the combination will exceed 100% effectiveness for genotype *2, but will not greatly improve the effectiveness for genotypes *3, *4 and *5 more than use of drug. D or E alone, and therefore, will not provide a greatly increased efficacy for 60% of the total population relative to the use of drug D or E alone.

In contrast, if two drugs are effective in treating different genotypes, use of the two drugs in combination can have an additive increase in efficacy for the entire population. For example, drug B, at 0.1 mg, has 10% or greater efficacy for genotypes *1, *3 and *4, while drug C, at 0.1 mg, has 10% or greater efficacy for genotypes *2 and *5, and therefore, the efficacy for the overall population is 31.3% for the combination of 0.1 mg drug B with 0.1 mg drug C.

In determining a suitable drug combination, optimized efficacy is combined with consideration of minimal toxicity. As used in this example, drugs A, B, C and D have low toxicity, while drug E has high toxicity. In Table I, the efficacies of compounds A through E are provided for each of five genotypes at four different dosage levels, and numerous dose combinations (dose comb) are presented. The mean efficacy (Mean eff), toxicity (tox) and toxicity index (ME/T) are provided for a variety of dose combinations, which included either zero, 0.1 or 1 mg of each drug. However, many of these combinations also have a significant amount of toxicity. For example, the combination of 0.1 mg A, 0.1 mg B, 0.1 mg C, 0.1 mg D and 1 mg E, is effective for 61% of the total patient population, but this combination is also toxic for 50% of the total patient population. Knowledge of drug overlap and drug toxicity enables maximal coverage of the total patient population without significant toxicity, such as shown for the two drug combinations: 0.1 mg A, 0.1 mg B, 0.1 mg C and 0.1 mg D, which is effective for 49.3% of the total patient population while not being toxic; and 0.1 mg B, 0.1 mg C and 0.1 mg D, which is effective for 46.1% of the total patient population while not being toxic.

Without knowledge of the efficacy of a drug on the various genotypes in a population, the efficacy of each drug is only reported in terms of the efficacy for the overall population. Knowing only the efficacy for the overall population, the efficacy of a combination drugs cannot be determined. That is, two drugs may be combined, but lack of knowledge of the efficacy of a drug on the various genotypes in a population can yield any possible result varying from greatly increased efficacy to in zero increase in efficacy. Maximal increase in efficacy of a combination of drugs is dependent on, for example, minimal drug overlap, which is determined by knowledge of the efficacy of each drug for each genotype.

TABLE I

| | Efficacy at | | | | Efficacy at | | | | Efficacy at | | | | Efficacy at | | | | Efficacy at | | | |
|---|---|---|---|---|---|---|---|---|---|---|---|---|---|---|---|---|---|---|---|---|
| | Indicated Dose | | | | Indicated Dose | | | | Indicated Dose | | | | Indicated Dose | | | | Indicated Dose | | | |
| | (%) | | | | (%) | | | | (%) | | | | (%) | | | | (%) | | | |
| | Drug A | | | | Drug B | | | | Drug C | | | | Drug D | | | | Drug E | | | |
| | 0.1 | 1 | 10 | 50 | 0.1 | 1 | 10 | 50 | 0.1 | 1 | 10 | 50 | 0.1 | 1 | 10 | 50 | 0.1 | 1 | 10 | 50 |
| Gene | mg | mg | mg | mg | mg | mg | mg | mg | mg | mg | mg | mg | mg | mg | mg | mg | mg | mg | mg | mg |
| 1* | 10 | 50 | 90 | 95 | 10 | 25 | 35 | 45 | 2 | 5 | 20 | 50 | 50 | 90 | 95 | 97 | 1 | 3 | 5 | 8 |
| 2* | 3 | 10 | 50 | 80 | 2 | 5 | 20 | 50 | 30 | 60 | 85 | 90 | 20 | 23 | 25 | 25 | 95 | 97 | 98 | 98 |
| 3* | 2 | 5 | 20 | 50 | 10 | 50 | 90 | 95 | 0.5 | 3 | 10 | 30 | 0.1 | 1 | 10 | 50 | 10 | 30 | 70 | 90 |
| 4* | 0 | 0 | 0 | 0 | 50 | 90 | 95 | 97 | 1 | 3 | 15 | 20 | 3 | 10 | 30 | 70 | 0.5 | 3 | 20 | 50 |
| 5* | 1 | 3 | 5 | 8 | 1 | 3 | 10 | 30 | 50 | 90 | 95 | 97 | 1 | 1 | 1 | 1 | 2 | 10 | 50 | 70 |

Cumulative efficacies for the selected representative dose combinations listed

| Dose | | | | | | | | | |
|---|---|---|---|---|---|---|---|---|---|
| comb | .1/.1 | .1/.1/.1 | .1.1.1.1 | .1.1.1.1.1 | .1/0/.1 | 0/.1/.1 | 00.1.1 | 000.1.1 | 0.1.1.1/0 |
| Geno | | | | | | | | | |
| type | | | | | | | | | |
| 1* | 20 | 22 | 72 | 73 | 12 | 12 | 52 | 51 | 62 |
| 2* | 5 | 35 | 55 | 150 | 33 | 32 | 50 | 115 | 52 |
| 3* | 12 | 12.5 | 12.6 | 22.6 | 2.5 | 10.5 | 0.6 | 10.1 | 10.6 |
| 4* | 50 | 51 | 54 | 54.5 | 1 | 51 | 4 | 3.5 | 54 |
| 5* | 2 | 52 | 53 | 55 | 51 | 51 | 51 | 3 | 52 |
| MEAN | 17.8 | 34.5 | 49.3 | 61 | 19.9 | 31.3 | 31.5 | 33.5 | 46.1 |
| EFF | | | | | | | | | |
| TOX | 0 | 0 | 0 | 50 | 0 | 0 | 0 | 15 | 0 |
| TI = M | high | high | high | 1.22 | high | high | high | 2.23 | high |
| E/T | | | | | | | | | |

| Dose | | | | | | | | |
|---|---|---|---|---|---|---|---|---|
| comb | 00.1.1.1 | .1.1.1.11 | .1.1.111 | 1.1.1111 | 0.11111 | 11111 | 0/.1/0/1/0 | 0/10/.1/50/0 |
| Geno | | | | | | | | |
| type | | | | | | | | |
| 1* | 53 | 75 | 115 | 118 | 133 | 173 | 33 | 134 |
| 2* | 145 | 152 | 155 | 185 | 188 | 195 | 25 | 75 |
| 3* | 10.6 | 42.6 | 43.5 | 46 | 86 | 89 | 11 | 140.5 |
| 4* | 4.5 | 57 | 64 | 66 | 106 | 106 | 60 | 166 |
| 5* | 53 | 63 | 63 | 103 | 105 | 107 | 2 | 61 |
| MEAN | 44.2 | 67.5 | 74.1 | 82.4 | | 97.8 | 26.2 | 87.2 |
| EFF | | | | | | | | |
| TOX | 45 | 52 | 70 | 106 | | 181 | 0 | 140.5 |
| TI =M | 0.98 | 1.3 | 1.06 | 0.78 | | 0.54 | high | 0.62 |
| E/T | | | | | | | | |

### Example II

Two Drugs with Additive Efficacies for Two Variably Populated Genotypes

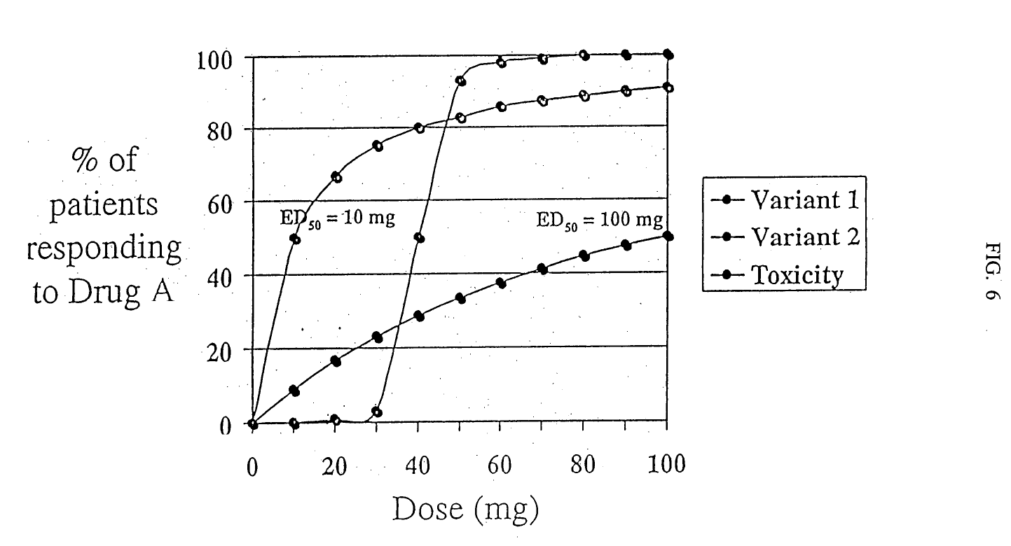

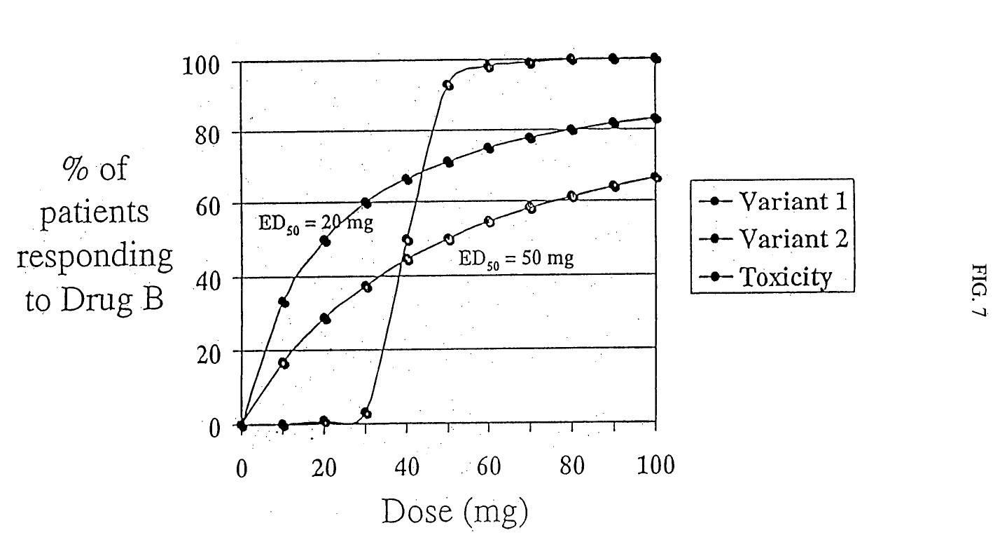

In another example, knowledge of the efficacy of each drug can be used to prepare a drug combination even in the absence of knowledge of the exact distribution of different genotypes in the total patient population. In this example, two drugs, A and B have different efficacies for two different SNP variant groups, V1 and V2, of the total patient population. Once again, these drugs will be considered to be independent of one another, and therefore, the efficacies of the two drugs for either SNP variant are additive. FIGS. 6 and 7 show the dose-response relationships and toxicity thresholds for drugs A and B, respectively. Both drugs have a threshold toxicity of 30 mg, and for this example, the threshold toxicities are additive. For example, 20 mg of drug A in combination with 20 mg of drug B has the same toxicity as 40 mg of drug A or drug B. Thus, the maximum sum of drugs A and B that can be used in a combination while maintaining low toxicity is 30 mg.

Table II shows the efficacies of different drug dose amounts of drugs A and B and of two combinations of A and B, for six different population distributions of SNP variants V1 and V2. Without knowledge of the exact distribution of the SNP variant groups in the total patient population, the combination with the highest mean response represents the combination effective for the maximum amount of the total patient population. The combination of 10 mg of drug A and 20 mg of drug B shows the highest mean response, which implies that this combination is most likely to be the most effective combination for treatment of any unknown population distribution of SNP variant groups V1 and V2. The combination of 10 mg of drug A and 20 mg of drug B also shows the lowest variability (% CV), meaning that this combination has the most consistent efficacy over the entire spectrum of possible population distributions of SNP variant groups V1 and V2.

TABLE II

| VARIANT | | MONOTHERAPY (DRUG dose) | | | | | | COMBINATIONS | |
|---|---|---|---|---|---|---|---|---|---|
| V1 | V2 | A₁₀ | A₂₀ | A₃₀ | B₁₀ | B₂₀ | B₃₀ | A₁₀B₂₀ | A₂₀B₁₀ |
| 100 | 0 | 50.0 | 66.7 | 75.0 | 16.7 | 28.6 | 37.5 | 78.6 | 83.4 |
| 80 | 20 | 41.8 | 56.7 | 64.6 | 20.1 | 32.9 | 42.0 | 74.7 | 76.8 |
| 60 | 40 | 33.6 | 46.7 | 54.2 | 23.3 | 37.2 | 46.5 | 70.8 | 70.0 |
| 40 | 60 | 25.5 | 36.7 | 43.9 | 26.7 | 41.4 | 51.0 | 66.9 | 63.4 |
| 20 | 80 | 17.3 | 26.7 | 33.5 | 30.0 | 45.7 | 55.5 | 63.0 | 56.7 |
| 0 | 100 | 9.1 | 16.7 | 23.1 | 33.3 | 50.0 | 60.0 | 59.1 | 50.0 |

| | | | | | | | | |
|---|---|---|---|---|---|---|---|---|
| MEAN | 29.6 ± 15.3 | 41.7 ± 18.7 | 49.1 ± 19.4 | 25.0 ± 6.2 | 39.3 ± 8.0 | 48.8 ± 8.4 | 68.9 ± 7.3 | 66.7 ± 12.5 |
| RESPONSE | | | | | | | | |
| CV (%) | 51.7 | 44.8 | 39.5 | 24.8 | 20.4 | 17.2 | 10.6 | 18.7 |

Although the invention has been described with reference to the disclosed embodiments, it should be understood that various modifications can be made without departing from the spirit of the invention. Accordingly, the invention is limited only by the following claims.

We claim:

1. A composition of compounds effective for treating a pathology, said composition comprising at least two compounds that modulate the activity of one or more target molecules associated with one or more Single Nucleotide Polymorphisms (SNPs), wherein each compound modulates the activity of at least one target molecule associated with one or more SNPs, and wherein said combination is effective for at least one patient having said pathology.

2. The composition of claim 1 wherein said combination is effective for at least 1% of patients having said pathology.

3. The composition of claim 1 wherein said combination is effective for at least 25% of patients having said pathology.

4. The composition of claim 1 wherein said combination is effective for at least 50% of patients having said pathology.

5. The composition of claim 1 wherein said combination is effective for at least 75% of patients having said pathology.

6. The composition of claim 1 wherein said combination is effective for at least 90% of patients having said pathology.

7. The composition of claim 1 wherein said compound interacts with said target molecule at a position corresponding to said SNP or at a position corresponding to a residue encoded by a codon comprising said SNP.

8. The composition of claim 1 wherein said composition comprises at least three compounds.

9. The composition of claim 1 wherein said composition comprises at least four compounds.

10. The composition of claim 1 wherein said composition comprises at least five compounds.

11. The composition of claim 1 wherein said composition comprises at least six compounds.

12. A composition of compounds effective for treating a pathology, said composition comprising at least two compounds that modulate the activity of at least one target molecule associated with at least one SNP, wherein said combination is effective for at least one patient having said pathology.

13. The composition of claim 12 wherein the modulation effect of each compound is correlated with the presence of the same SNP associated with the same target molecule.

14. The composition of claim 12 wherein the modulation effect of each compound is correlated with the presence of a mutually exclusive SNP associated with the same target molecule.

15. The composition of claim 14 wherein said mutually exclusive SNPs occur in a single patient.

16. The composition of claim 14 wherein said mutually exclusive SNPs occur in a plurality of patients.

17. The composition of claim 12 wherein the modulation effects of said compounds are correlated with the presence of a SNP on at least two target molecules.

18. The composition of claim 17 wherein at least one target molecule is associated with at least two SNPs.

19. The composition of claim 18 wherein at least two target molecules are associated with at least two SNPs.

20. A composition of compounds effective for treating a pathology, said composition comprising at least two compounds that modulate the activity of a single target protein associated with one or more SNPs, wherein said combination is stably effective for at least one patient having said pathology.

21. The composition of claim 20 wherein the modulation effect of each compound is correlated with the presence of the same SNP associated with said target protein.

22. The composition of claim 20 wherein the modulation effect of each compound is correlated with the presence of a mutually exclusive SNP associated with said target protein.

23. The composition of claim 20 wherein said composition comprises at least three compounds.

24. The composition of claim 20 wherein said composition comprises at least four compounds.

25. The composition of claim 20 wherein said combination is stably effective for at least 50% of patients having said pathology.

26. The composition of claim 20 wherein said combination is stably effective for at least 75% of patients having said pathology.

27. The composition of claim 20 wherein said combination is stably effective for at least 90% of patients having said pathology.

28. A method of increasing overall treatment efficacy for a given population of patients having a pathology, comprising:
(a) analyzing compounds known or suspected of modulating the activity of at least one target molecule corresponding to said pathology for efficacy correlated with the presence of a SNP associated with said target molecule(s); and
(b) selecting, for treatment of said patients, a combination of at least two of said compounds that exhibit the highest overall mean response of all dosing options in said population of patients.

29. The method of claim 28 wherein said combination is effective for at least 25% of said patients.

30. The method of claim 28 wherein said combination is effective for at least 50% of said patients.

31. The method of claim 28 wherein said combination is effective for at least 75% of said patients.

32. The method of claim 28 wherein said combination is effective for at least 90% of said patients.

33. A method of maximizing overall population efficacy of treatment for a particular pathology, comprising:
(a) determining the efficacies of a plurality of compounds known or suspected of treating said pathology;
(b) determining the toxicity of said plurality of compounds; and
(c) selecting from said plurality of compounds a combination of compounds that is minimally toxic and is effective for at least 1% of the total patient population having said pathology.

34. A method of formulating a pharmaceutical composition for treating a particular pathology in a population of patients having said pathology, comprising:
(a) measuring a correlation of genetic variation of a target molecule in said population with patient response to at least one compound known or suspected to treat said pathology; and
(b) selecting at least two compounds that provide the greatest percentage of efficacy in said patient population, wherein said percentage is at least 1% of the total patient population having said pathology.

35. The method of claim 34 wherein said composition comprises at least three compounds.

36. The method of claim 34 wherein said composition exhibits minimal toxicity.

37. The method of claim 34 wherein said composition exhibits no toxicity.

38. A genotypically-facilitated method of treating one or more patients having a pathology, comprising:
(a) analyzing a therapeutic target molecule in a population of patients having said pathology to detect SNPs associated therewith;
(b) selecting a plurality of compounds having therapeutic efficacies correlated with the presence of at least one SNP associated with said target molecule; and
(c) administering said plurality of compounds to a patient in said population;
wherein said combination is effective for at least 1% of the total patient population having said pathology.

39. The method of claim 38 wherein said plurality of compounds is administered to said patient simultaneously or proximately to one another.

40. A method of formulating a therapeutic composition to treat a pathology, comprising:
(a) analyzing a target molecule in a patient population to detect SNPs associated therewith; and
(b) selecting a plurality of compounds having therapeutic efficacies correlated with the presence of at least one SNP associated with the target molecule, wherein said plurality is effective for at least 1% of the total patient population having said pathology.

41. A method of selecting an optimized therapeutic composition to treat a pathology, comprising selecting a combination of at least two compounds that exhibits the highest overall mean response of all dosing options and exhibits the lowest variation in response across different population groups, wherein said combination is effective for at least 1% of the total patient population having said pathology.

42. The method of claim 41 wherein a total dosage of said composition does not exceed a threshold for toxicity.

43. A method of preparing an optimized pharmaceutical composition, comprising identifying a combination of at least two compounds that exhibit maximal population efficacy across all known SNPs associated with a particular target molecule correlated with a pathology, wherein said combination exhibits minimal toxicity.

44. The method of claim 43 wherein said combination is effective for at least 25% of patients having said pathology.

45. The method of claim 43 wherein said combination is effective for at least 50% of patients having said pathology.

46. The method of claim 43 wherein said combination is effective for at least 75% of patients having said pathology.

47. The method of claim 43 wherein said combination is effective for at least 90% of patients having said pathology.

48. A method of optimizing therapeutic treatment of a pathology, comprising selecting a combination of compounds that minimize overlap of drug efficacy in patients having said pathology, wherein said combination of compounds exhibits minimal toxicity.

49. The method of claim 48 wherein said combination is effective for at least 25% of patients having said pathology.

50. The method of claim 48 wherein said combination is effective for at least 50% of patients having said pathology.

51. The method of claim 48 wherein said combination is effective for at least 75% of patients having said pathology.

52. The method of claim 48 wherein said combination is effective for at least 90% of patients having said pathology.

53. A method of treating a sub-population of patients having a particular pathology, comprising:
(a) identifying a sub-population of patients having at least one known SNP from all patients exhibiting said pathology; and
(b) administering to said sub-population a composition comprising at least one therapeutic compound having an efficacy correlated with the presence of said SNP.

54. A method of treating a sub-population of patients having a particular pathology, comprising:
(a) identifying a sub-population of patients having a known SNP profile from all patients exhibiting said pathology; and
(b) administering to said sub-population a composition comprising at least one therapeutic compound having an efficacy correlated with said SNP profile.

55. A method of preparing a combination of compounds for treating one or more patients having a pathology, wherein said combination of compounds has increased efficacy and/or reduced toxicity, relative to any individual compound, in a greater portion of a population of patients having said pathology, comprising:
(a) correlating the efficacy and/or toxicity of a first compound with the presence of one or more SNPs;
(b) correlating the efficacy and/or toxicity of a second compound with the presence of one or more SNPs; and
(c) calculating the efficacy and/or toxicity of a combination of said first compound and said second compound on said population of patients;
wherein said combination is effective for at least 1% of the total patient population having said pathology.

56. The method of claim 55, further comprising the steps of:
(d) correlating the efficacy and/or toxicity of a third compound with the presence of one or more SNPs; and
(e) calculating the efficacy and/or toxicity of a combination of said first compound, said second compound and said third compound on said population of patients.

57. An algorithm for determining the efficacy and/or toxicity of a combination of two or more compounds for a population of patients having a pathology, comprising the steps of:
(a) correlating the efficacy and/or toxicity of each of said two or more compounds with the presence of one or more SNPs; and
(b) combining the correlations of the efficacy and/or toxicity of each of said two or more compounds with the presence of one or more SNPs;
whereby the efficacy and/or toxicity of said combination of compounds for said population of patients is calculated.

58. A method of treating a patient having a pathology with a combination of therapeutic compounds having increased efficacy and/or reduced toxicity, relative to any individual compound, in a greater portion of a population of patients having said pathology, comprising the steps of:
(a) correlating the efficacy and/or toxicity for each of two or more compounds with one or more SNPs;
(b) calculating the efficacy and/or toxicity of administering said two or more compounds on said population of patients; and
(c) administering said two or more compounds to said patients;
wherein said two or more compounds are effective for at least 1% of the total patient population having said pathology.

59. The method of claim 57 wherein each of said two or more compounds is administered to said patient simultaneously or proximately to one another.
